(19) United States
(12) Patent Application Publication
(10) Pub. No.: US 2020/0222264 A1
(43) Pub. Date: Jul. 16, 2020

(54) APPARATUS FOR BACK DECOMPRESSION WITH VARIABLE SIZED WAISTS

(71) Applicant: Joseph Golightly, State College, PA (US)

(72) Inventor: Joseph Golightly, State College, PA (US)

(21) Appl. No.: 16/742,971

(22) Filed: Jan. 15, 2020

Related U.S. Application Data
(60) Provisional application No. 62/792,949, filed on Jan. 16, 2019.

(51) Int. Cl. A61H 1/02; A61F 5/02

(52) U.S. Cl. CPC A61H 1/0218; A61H 1/0292; A61H 2201/1623; A61F 5/02

(57) ABSTRACT

An apparatus for relieving spinal compression without inverting the user. The apparatus is fully adjustable for those of all sizes and provides much needed back relief to those with compressed and burdened spinal columns. The apparatus provides handles for the user to press down on, pushing the shoulders upward, and allowing the arms to take away some stress from the back.

### CROSS REFERENCES

This application claims the benefit of U.S. Provisional Application No. 62/792,949, filed Jan. 16, 2019, which application is incorporated herein by reference.

### BACKGROUND OF THE INVENTION

Back pain is a common issue for those of all ages, and back pain especially plagues the elderly. A lot of the pain comes from spinal compression that has occurred from the body constantly fighting a losing battle with gravity. Vertebrae can become dislocated, or the tissue between vertebrae gets overly compressed or worn away. The spine needs to have some of the downward weight taken off.

Inversion tables have been used to reduce compression of the spine. The inversion table uses gravity to decompress the spine through holding the body upside down. Several problems exist relating to inversion tables, including blood becoming pooled in the head. Ringing or other irritations can happen while being held upside down. Inversion tables also take up a large amount of space and can be difficult to move and expensive to transport. Described herein are improved methods and devices for decreasing spinal decompression that do not rely on the use of an inversion table.

Lower back pain is a frequent issue for people of all ages, and lower back pain is especially common for those in middle-age and on into later life. The widely recognized reason for this pervasive form of pain is the fact that humans are erect standing and walking beings. As a result, the force of gravity is constantly pulling down on all parts of the body, and the lower ⅓ of the spinal column especially is a focal point of that constant tugging force. The spine is the bone and flexible disc system that holds the upper body upright, while it also provides bending and twisting flexibility. The pull of gravity acts to try to compress the vertebrae and disc system over time, and the most affected region of compression occurs in the lower back. The nerve bundles and blood vessels that travel through the spinal column become subjected to this compression force as well, to adverse effects. Squeezed and pinched nerves become a source of chronic pain or numbness under these circumstances, and critical blood flow becomes restricted. These factors can lead to even more severe spinal degeneration and nerve damage over time, if left untreated.

Inversion tables have been used to counteract compression of the spine. The inversion table uses gravity to decompress the spine through holding the body in an upside-down position. Several problems exist relating to inversion tables, including blood becoming pooled in the head. Ear ringing or other irritations can happen while being suspended upside down. Inversion tables also take up a large amount of space, can be difficult to move, and expensive to transport. Described herein are improved methods and devices for counteracting spinal compression that do not rely on the use of an inversion table.

In this embodiment, the apparatus provides an opportunity for users to apply a decompressive force to their spinal column, without the drawbacks involved with inversion. In this example, the apparatus fits snugly around the user's midsection, functionally at the narrowest circumference around the torso, in the area that resembles the hourglass shape of the torso as viewed from the front or rear of the user (i.e., waist area). In this example, the user applies a downward force with their hands and arms through component handles of the apparatus, to affect a decompressive force imparted to their spinal column.

### SUMMARY OF THE INVENTION

In some embodiments, an apparatus includes: a belt; a handle attached to the belt; and an adjustable fastening system included on the belt. In some embodiments, the adjustable fastening system is a teeth and channel fastening system. In some embodiments, the belt is made of a durable plastic. In some embodiments, the belt has a conical shape with the top being smaller than the bottom. In some embodiments, the belt only comes around the back and sides. In some embodiments, the adjustable fastening system is a hook-and-loop fastening system. In some embodiments, the handle is made of a plastic material. In some embodiments, the belt is made of a fibrous material. In some embodiments, the belt is made of a plastic material. In some embodiments, the belt and the handle are molded plastic. In some embodiments, the belt and the handle are molded together.

In some embodiments, an apparatus comprises: a handle and a slot on the handle. In some embodiments, the slot is sized to fit normal widths and heights of a belt. In some embodiments, the apparatus is made of a durable plastic. In some embodiments, the handle is configured to fit a human hand. In some embodiments, the handles slide onto the belt.

In some embodiments, a method of spinal decompression includes: putting on spinal decompression belt; placing hands on handles of spinal compression belt; pressing down on handles with handles while keeping shoulders high; and bringing chest upward. In some embodiments, the method includes the step of laying down after releasing the handles. In some embodiments, a user repeats the method multiple times throughout the day. In some embodiments, the method includes the step of sliding a handles on a belt to create the spinal decompression belt.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an exemplary illustration of an embodiment of the adjustable size belt.

FIG. 2 is an exemplary illustration of an embodiment of a fastening system.

FIG. 3 is an exemplary photograph of a prototype of one embodiment of a slip-on handle.

FIG. 4 is an exemplary photograph of a prototype of one embodiment of a slip-on handle.

FIG. 5 is an exemplary photograph of a prototype of one embodiment of a slip-on handle.

FIG. 6 is an exemplary illustration of a belt and a handle loop.

FIG. 7 is an exemplary illustration of one embodiment of a spinal decompression belt with slip-on handles.

FIG. 8 is an exemplary illustration of one embodiment of the shape of a spinal-decompression belt.

FIG. 9 is an exemplary illustration of one embodiment of a slip-on handle.

FIG. 10 is an exemplary illustration of one embodiment of a spinal decompression belt with fixed handles.

FIG. 11 is an exemplary illustration of one embodiment of a spinal decompression belt with slip-on handles.

FIG. 12 is an exemplary illustration of one embodiment of a back-latching mechanism.

FIG. 13 is an exemplary illustration of one embodiment of a latching mechanism.

### DETAILED DESCRIPTION OF THE INVENTION

In some embodiments, the apparatus provides an opportunity for users to decompress their spinal column, without the drawbacks involved with inversion. In some embodiments, the apparatus fits around the user's midsection (e.g., waist area). The handles can be placed near a user's frontal hip bones (e.g. near the anterior portion of the iliac crest). The user can provide force in a caudal direction on the handles (e.g. hold the handles with their hands and push downwards towards the feet). This pushing action can provide an upwards force on the shoulders and a downwards force on the hips. This bidirectional force can relieve pressure between individual vertebrae, e.g., bringing vertebrae up, relieving a large amount of pressure from the spine, and redistributing the pressure to the arms temporarily. The action can be done daily, weekly, or whenever irritation arises.

In one embodiment, the adjustable apparatus is a belt. The belt can have two handles attached near the sides of the belt, to sit near the front hip bones. In this embodiment, the belt will be adjustable. The adjustable nature can be done through a teeth and channel system or through some other measure. In the embodiment including a teeth and channel system, the teeth and channel fastener may be accompanied by a release tab to loosen the belt after use. In this embodiment, the teeth and channel system will work by having two main pieces: a male piece and a female piece. In this embodiment, the male piece will have protruding pieces that will lock into channels in the female piece. In this embodiment, the male teeth will have a slight gradient on the side facing the entrance to the female piece, and the opposing side will have no gradient. In this embodiment, the teeth having a gradient on one side but not the other, which will allow for the pieces to slide easily together, but the pieces will require the pulling of the release tab to come apart. In this embodiment, the female channels will be slots that match the teeth with a corresponding gradient and lack of gradient in the valleys of the channels. In one embodiment, the whole belt is made of a durable plastic. In another embodiment, the belt is made of sown together fiber.

In some embodiments of this embodiment, the belt is conical shaped in which the part of the belt higher up on the body is smaller than the part below. This specific embodiment can ensure the belt has a better hold of the body to allow for the belt not to slip during the decompression action.

Another embodiment of this embodiment does not rap all the way around the body. This embodiment involves only wrapping around the back and sides of a user. In this embodiment, the belt still fastens on the body, but the belt does not have a front portion.

In another embodiment, the adjustable apparatus includes two removable handles. In this embodiment, the user slides the two handles on a belt. In this embodiment, the handles include a slot perfectly sized for the belt to slide through. In this embodiment, the user attempts to, but does not have to, place the handles near the sides of the body. Once the belt is slid through the slots of the handles and tightened, the user can utilize the pressure system to decompress the back. In one embodiment, the handles are made of a durable plastic.

In both of the above described embodiments, the user decompresses the spine through the same action of pressing down on the handles with locked elbows. The motion can alleviate some of the stress on the back and provide some relief for back pain caused by a compressed spinal column.

In some embodiments, the belt is intended for larger individuals. In some embodiments, the belt has two main parts: a left side and a right side. Each side can include one side to a male-female fastener system in the back or front of the piece. When the fastener system is locked in, the belt may have a hook-and-loop fastener in the front. In some embodiments, the belt has two removable handles that are slid onto the belt to complete the intended purpose. In some embodiments, the handles have padding meant to rest on the user's sides.

In some embodiments, the optimal use of the belt occurs when the user uses the belt several times a day with each session lasting 3 to 10 minutes. In some embodiments, the use of the belt is followed by the user laying down.

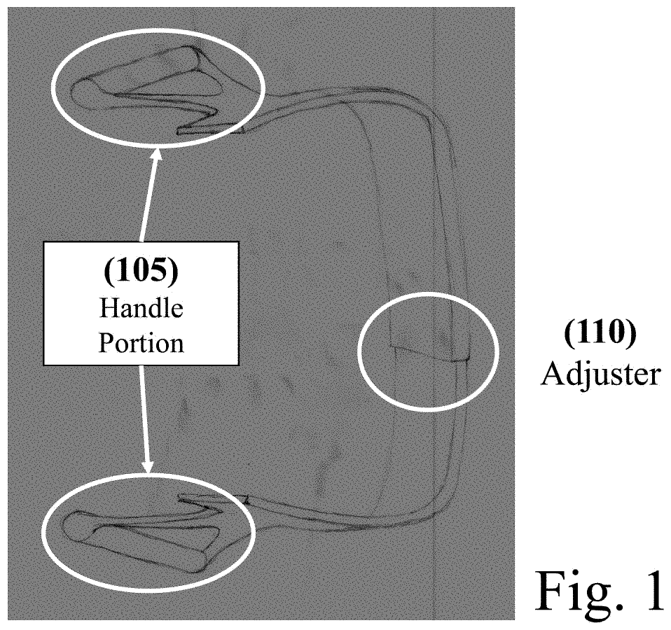

FIG. 1 is an illustration of an embodiment of the apparatus that allows users of various sizes to relieve back pain through an adjustable apparatus. In this embodiment, the handle portions (105) are located near the sides of the adjustable belt. In this embodiment, the adjustable belt is adjustable through a teeth and channel adjuster (110). In this embodiment, the adjuster (110) can be on one side or both sides with one adjuster located in the back region. In this embodiment, the belt can synch-up on any size waste to allow anyone of any size to utilize the handles to decompress the spine and relieve stress on the back.

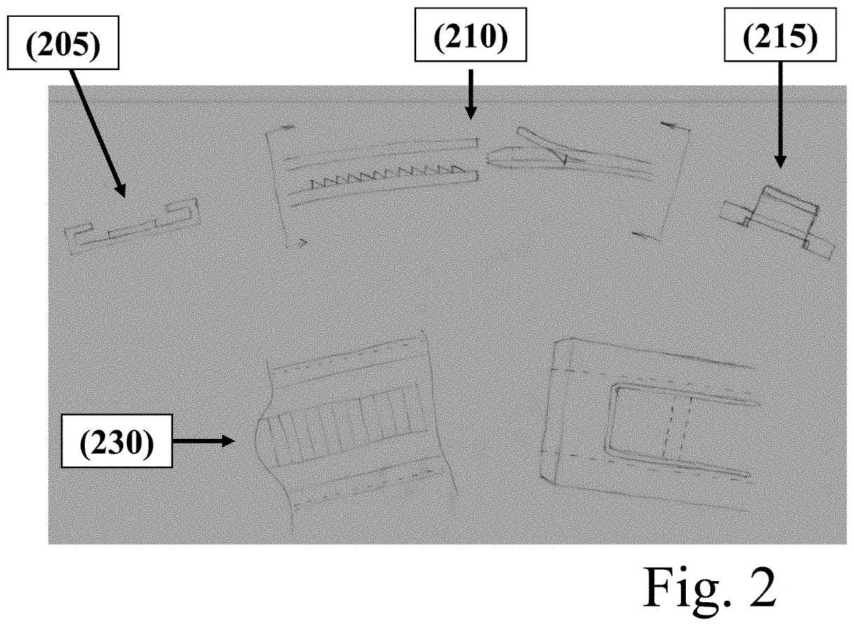

FIG. 2 is an illustration of an embodiment of the fastener system in an adjustable back relief belt. In this embodiment, the fastener system consists of two key pieces: a male piece (215) and a female piece (205). The male piece (215) in this embodiment has teeth which lock into slots, or channels, in the female piece (205). In this embodiment, the locking (210) of the male and female piece can be undone through pulling up of a tab and sliding the pieces apart. FIG. 2 conveys the male and female pieces of this embodiment from another perspective (230) as well.

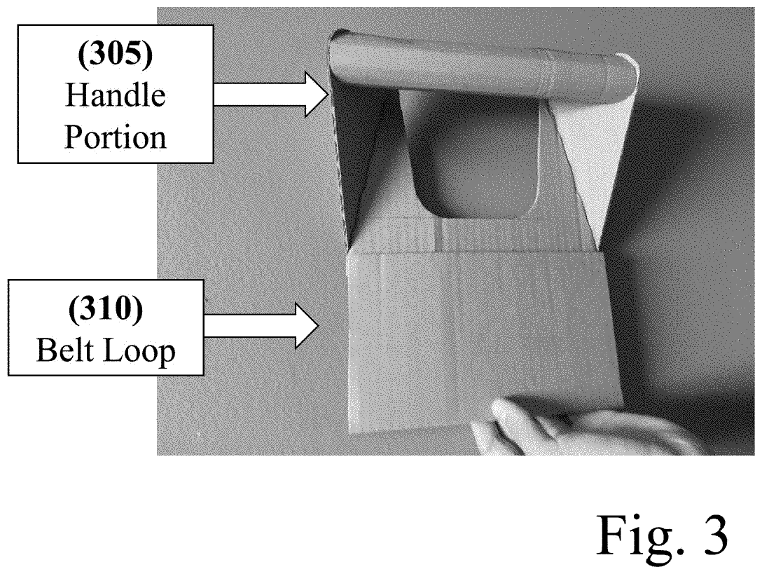

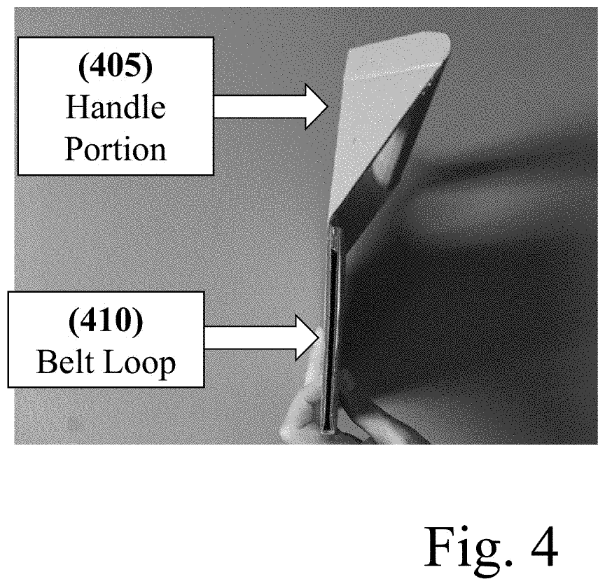

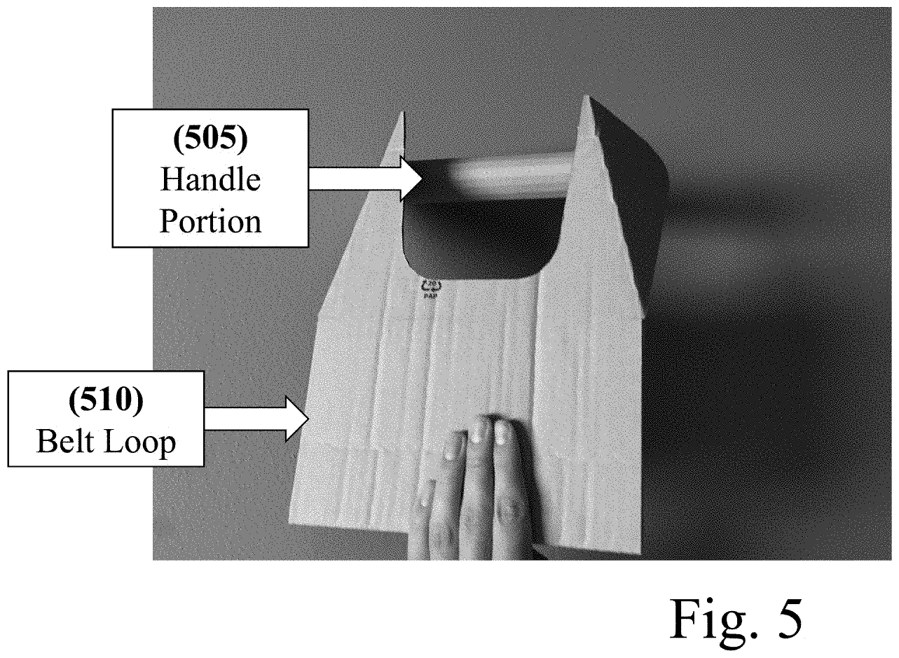

FIGS. 3, 4, and 5 all display one embodiment of the handle that connects to a belt. The three figures are different in the side shown through the figure. This embodiment is comprised of two main parts: a handle portion (305) (405) (505) and a belt slot or belt loop portion (310) (410) (510). In this embodiment, the handle slips onto any ordinary belt through the belt slot or belt loop (310) (410) (510). In this embodiment, once the handle is firmly fixed onto the belt, the user can tighten the belt until the belt is snug. When the belt is snug, the user can apply pressure on the handle or handles (305) (405) (505) to decompress the spine and provide relief for the user's back.

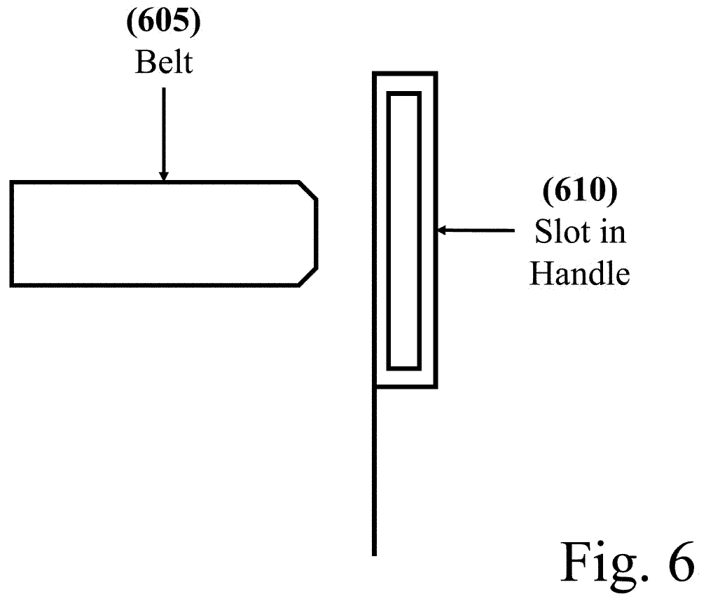

FIG. 6 depicts one embodiment of the slip-on handle for spinal decompression. In this embodiment, a slip-on handle such as the embodiment in FIGS. 3, 4, and 5 contains a slot (610). In this embodiment, the slot has adequate space to fit a belt (605). In this embodiment, the handle slides onto the belt (605) by slipping the belt (605) through the slot in the handle (610). In this embodiment, the user does so to place the handle or handles on the sides of the user. In this embodiment, the user tightens the belt, then use their arms to apply pressure to the handles, decompressing the spine and providing pain relief.

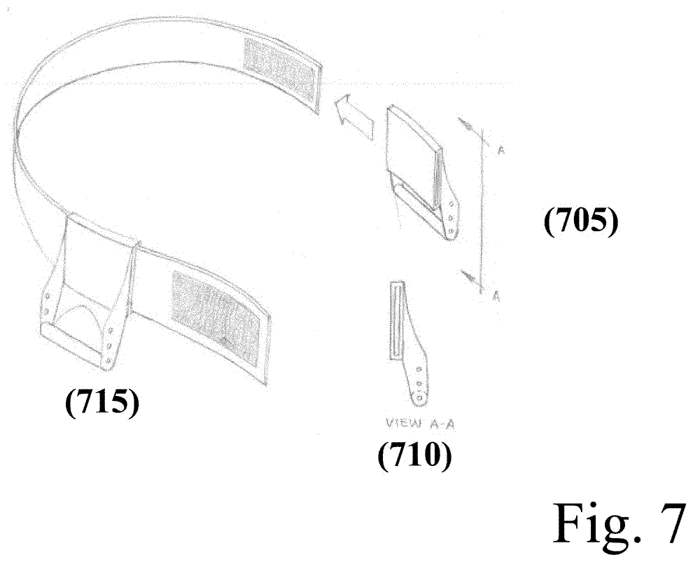

FIG. 7 depicts one embodiment of the belt with slip-on handles. In this embodiment, the belt (715) has hook-and-loop fasteners towards the front of the belt to allow the user to fix the belt on the body. The handles (705) can be slipped onto the belt and moved around to the desired area. In some embodiments, the handles (705) have a loop (710) especially sized for belts to be threaded through.

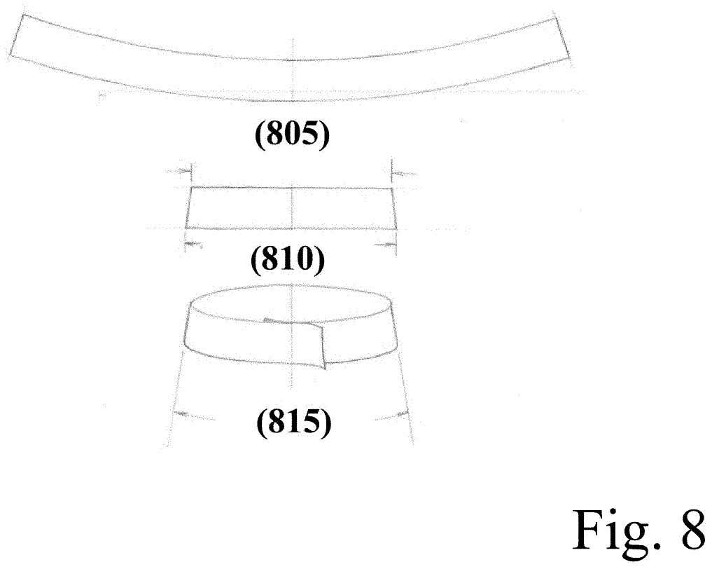

FIG. 8 conveys the shape of one embodiment of the belt. The belt can have a crescent shape (805) when the belt is laid on a flat surface. In some embodiments, the belt when in a closed loop (810) has a smaller circumference on top and a larger circumference on bottom. In some embodiments, the belt when closed and viewed from an isometric view (815) appears in a conical shape.

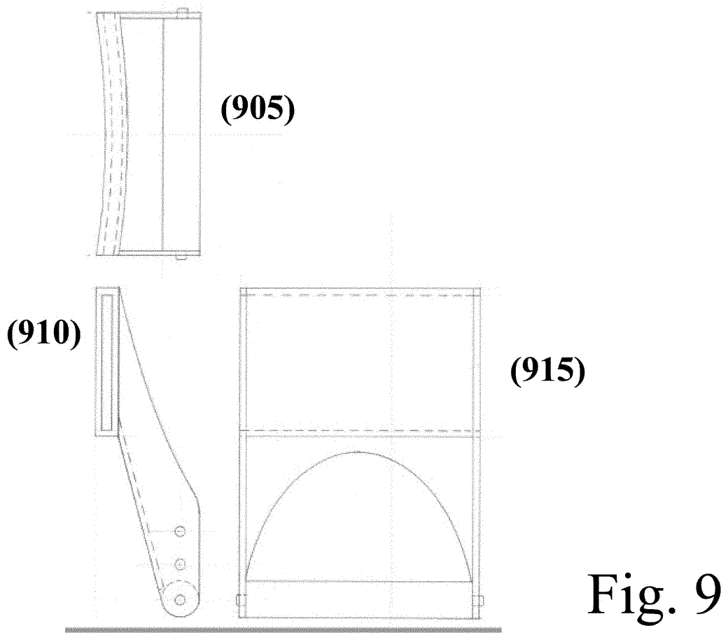

FIG. 9 portrays one embodiment of the removable handle. The top view (905), the side view (910), and the frontal view (915) show what this embodiment of the handle looks like.

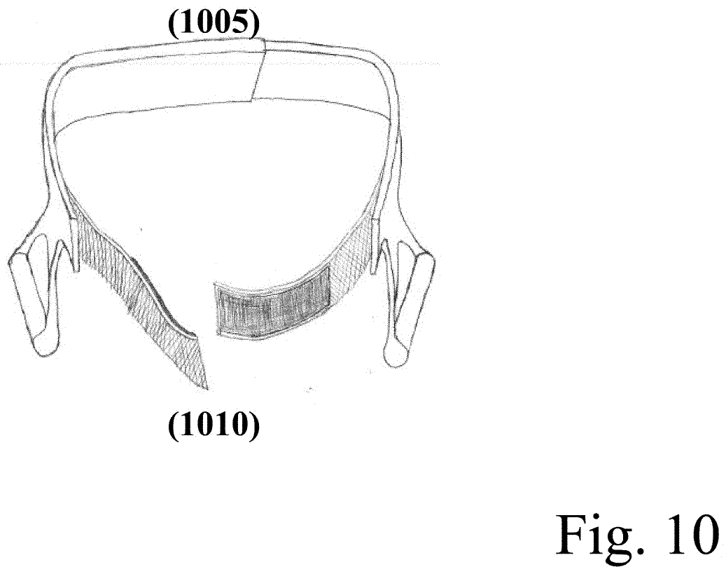

FIG. 10 depicts one embodiment of the belt with an integral handle variation. In this embodiment, the belt has an adjustable frame component (1005). In this embodiment, the belt is further equipped with a hook-and-loop fastener (1010).

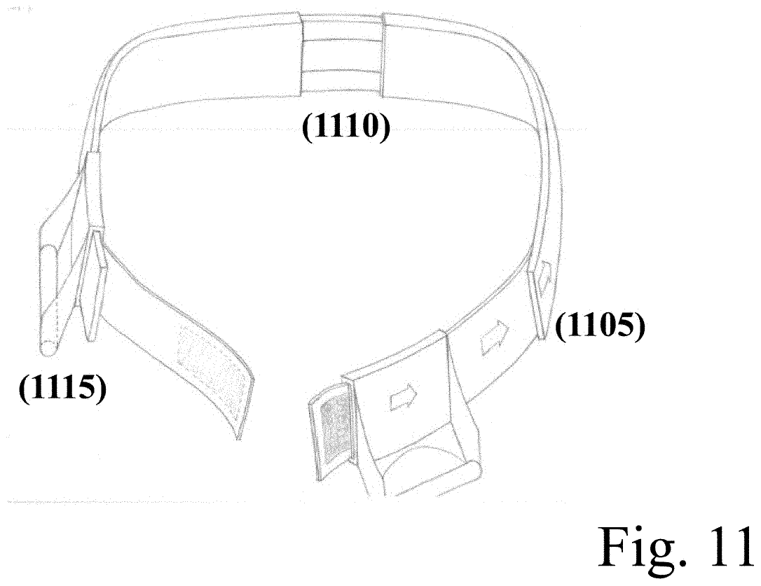

FIG. 11 depicts one embodiment of the belt with removable handles. In this embodiment, the handles (1115) can be slipped on (1105). In this embodiment, the belt has an adjustable frame component (1110) in the back and hook-and-loop fastener in the front.

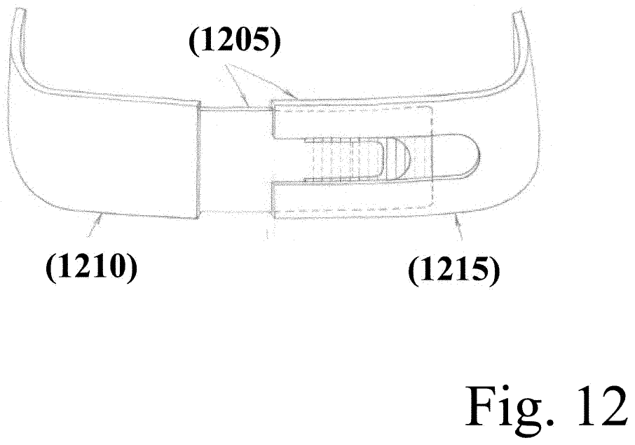

FIG. 12 depicts one embodiment of the adjustable-width two-piece rigid-plastic interconnected frame component from the rear. In this embodiment, the male frame half (1210) goes into the female frame half (1215), and the size adjusts through this fastener system (1205).

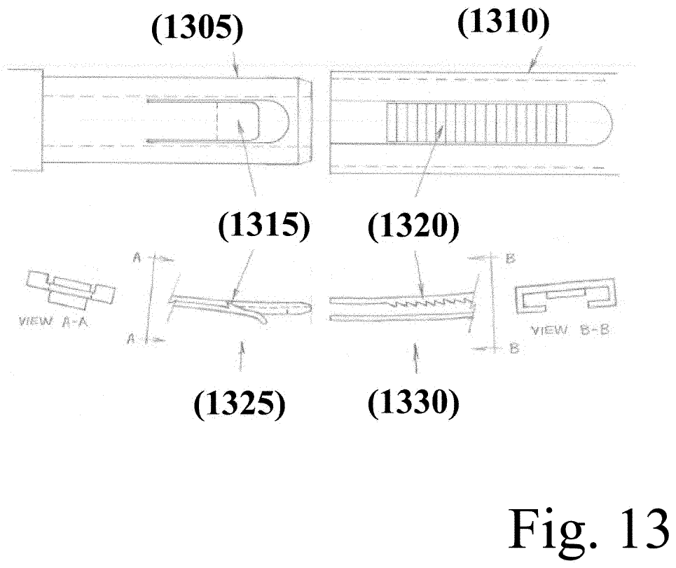

FIG. 13 depicts one embodiment of the fastener system. In this embodiment, the male tongue feature (1305) inserts into the female channel feature (1310). In this embodiment, the release tab with latching tooth (1315) allows the user to loosen the belt. In this embodiment, the tooth (1315) locks into the row of latching teeth (1320) when tightened. The release tab (1325) can be lifted away from the latching teeth (1330), and the belt parts can be separated.

### Example #1—Construction

In one example, the apparatus has an adjustable fastener, variable circumference belt component and two adjustable and removable handle components. In this example, the apparatus is comprised of a wide non-elastic flexible synthetic fiber (fabric) belt component that acts as the carrier and attachment structure for two removable molded plastic handle components. The handles incorporate a channel through which the belt is inserted, and once inserted on the belt, the handles can be adjusted to any position along the length of the belt. The handles incorporate height-adjustable hand grips which are grasped by the user. The belt is formed in a slight crescent shape, such that when looped in a circle, the belt displays a slight conical shape in profile. The top rim of the belt has a slightly smaller circumference than the bottom rim of the belt, as seen in profile. The belt ends have hook-and-loop fastening elements applied, male and female counterparts, or some other fastening method, to facilitate fastening the belt around the torso when in use.

### Example #1—Usage

This example of the apparatus is most suitable for people with typical approximate hourglass-shaped body types (“normal”). The apparatus, comprised of the belt component with the two handle components installed, is placed in position by wrapping it around the user's midsection. The belt ends overlap at the front of the torso, and are fastened snugly to each other by hook-and-loop fasteners, or by some other fastening method. Once the belt is applied around the user's waist area, the user can slide the handles along the belt to positions opposite of each other and adjacent to the user's left and right hip bones (i.e. near the anterior portion of the iliac crest). The user can then grasp the handles with their hands and apply a downward force to the belt. The belt remains snug around the user's waist area just above the hips, as the downward force is applied. The conical shape of the closed belt aids in the tightening action of the belt when put to use. As downward force is applied to the handles, the belt nudges down the torso slightly from the narrowest circumference of the user's waist towards the enlarging circumference of the hip area, reaching a stopping point. The stopping point is the foundation for the ensuing pressure that will be applied through the handles by the user's arms, to affect the decompression action. The hips are anatomically connected to the base of the spine, and provide the foundation for the pushing action. This downward pushing action by the arms applies a corresponding upward force on the shoulders, which are anatomically connected to the upper spinal column, lifting the top of the spinal column. As upper torso mass is redistributed temporarily to the arms, the spinal column length between the hips and the shoulders becomes slightly elongated. This bidirectional force effectively acts to impart a decompressive force to the spinal column. The activity can be engaged in several times daily, once a day, weekly or whenever lower back pain becomes an issue. Normal length of time to engage in the activity is 3 to 10 minutes per session. The activity can be performed while standing vertically or during easy walking, and it can be performed more customarily with the body in the prone position, lying on the floor or on a bed. The activity of decompressing the spinal column with this apparatus will have the longest lasting effects when performed in bed, immediately before going to sleep. The decompression effects will remain longer when the user remains in the prone position afterward, as opposed to returning immediately to the gravity compressing upright position.

### Example #2—Construction

In one example, the apparatus has a two-piece rigid-plastic frame component, an adjustable length securing belt component, and two adjustable and removable handle components. The two-piece frame component is adjustable for width across its interconnected midsection. The adjustability can be done through a sliding channel and interlocking-tooth latching system or through some other measure. In this example, the apparatus has an adjustable width U-shaped frame structure comprised of left and right side sliding and interlocking frame pieces made of rigid molded plastic material. The adjustability function is created by having a molded insertion tongue feature on the male frame half, which corresponds to and inserts into a molded channel feature in the female frame half. For the interlocking function, there is a linear row of successive latching teeth centered in the channel on the female frame half, and a corresponding latching tooth molded as a feature into a flexible release tab on the tongue of the male frame half. The teeth on both male and female halves of this latching system have gradient slopes facing the insertion side of each tooth, for easy insertion of male frame half into the female frame half, and flat, non-sloped, back sides on each tooth to facilitate a latching engagement. The male latching tooth on the flexible release tab is displaced by each successive tooth it passes in the female channel as the frame halves are inserted together, until the male tongue has been inserted to the desired depth. This tooth engagement holds the frame halves latched together at the chosen depth of insertion, until the release tab is lifted, disengaging the interlocking teeth and allowing the frame halves to slide apart, to remove the apparatus. In this example, the frame component has side arms which extend forward from the width-adjustable back section. Attached to the plastic frame arms are two wide non-elastic flexible synthetic fiber (i.e. fabric) belt components. The belt halves are bonded or otherwise attached to the inner surface of each of the two frame arms. The loose belt ends have hook-and-loop fastening elements applied, male and female counterparts, or some other fastening method, to facilitate fastening the belt around the torso when in use. In this example, the side arms of the frame component act as the carrier and attachment structure for the two removable molded-plastic handle components. The handles incorporate a channel through which the belt and frame arms are inserted, and once inserted on the frame, the handles can be adjusted to their proper usage position.

### Example #2—Usage

This example of the apparatus is suitable for all sizes and shapes of users, but is designed to be most suitable for people with extended waistlines, including people with portly waist circumferences, or possessing waist areas of greater circumference than the lower-torso hip area circumference (i.e. body types where the example #1 apparatus would not fit appropriately). The apparatus, comprised of the adjustable frame component, the fabric belt component and two installed handle components, is placed in position by first bringing the frame from the rear of the user and into contact with the user's lower back, with the two side arms extending straight forward. The frame arms height wise should be positioned just above the left and right hip bones. The user then pushes the two frame arms together, bringing them snug against the user's sides and displacing soft tissue until a “tight” position is reached without causing discomfort. The frame latching feature will “click” successively as the frame halves engage each other, and will hold position when the desired width is reached. (The release tab can be lifted to loosen or remove the frame component.) The user then attaches the fabric belt halves snuggly around the front of the torso by overlapping the belt ends and securing with the hook-and-loop fasteners, or other fastening method. This fitment secures the apparatus snuggly around the user's midsection circumference. The user can now slide the movable handles along the frame arms to positions opposite of each other and adjacent to the user's left and right hip bones (i.e. near the anterior portion of the iliac crest). The user can then grasp the handles with their hands and apply a downward force to the apparatus. The apparatus remains snug around the user's waist area just above the hips, as the downward force is applied. The grasp of the frame side arms to the sides of the user's torso above the hips provides a stopping point to downward force. The stopping point is the foundation for the ensuing pressure that will be applied through the handles by the user's arms, to affect the decompression action. The hips are anatomically connected to the base of the spine, and provide the foundation for the pushing action. This downward pushing action by the arms applies a corresponding upward force on the shoulders, which are anatomically connected to the upper spinal column, lifting the top of the spinal column. As upper torso mass is redistributed temporarily to the arms, the spinal column length between the hips and the shoulders becomes slightly elongated. This bidirectional force effectively acts to impart a decompressive force to the spinal column. The activity can be engaged in several times daily, once a day, weekly or whenever lower back pain becomes an issue. Normal length of time to engage in the activity is 3 to 10 minutes per session. The activity can be performed while standing vertically or during easy walking, and it can be performed more customarily with the body in the prone position, lying on the floor or on a bed. The activity of decompressing the spinal column with this apparatus will have the longest lasting effects when performed in bed, immediately before going to sleep. The decompression effects will remain longer when the user remains in the prone position afterward, as opposed to returning immediately to the gravity compressing upright position.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An apparatus comprising:
a belt;
a handle attached to the belt; and
an adjustable fastening system included on the belt.

2. An apparatus as in claim 1, wherein the adjustable fastening system is a teeth and channel fastening system.

3. An apparatus as in claim 1, wherein the belt is made of a durable plastic.

4. An apparatus as in claim 1, wherein the belt has a conical shape with the top being smaller than the bottom.

5. An apparatus as in claim 1, wherein the belt only comes around the back and sides.

6. An apparatus as in claim 1, wherein the adjustable fastening system is a hook-and-loop fastening system.

7. An apparatus as in claim 1, wherein the handle is made of a plastic material.

8. An apparatus as in claim 1, wherein the belt is made of a fibrous material.

9. An apparatus as in claim 1, wherein the belt is made of a plastic material.

10. An apparatus as in claim 1, wherein the belt and the handle are molded plastic.

11. An apparatus as in claim 1, wherein the belt and the handle are molded together.

12. An apparatus comprising:
a handle; and
a slot on the handle.

13. An apparatus as in claim 12, wherein the slot is sized to fit normal widths and heights of a belt.

14. An apparatus as in claim 12, wherein the apparatus is made of a durable plastic.

15. An apparatus as in claim 12, wherein the handle is configured to fit a human hand.

16. An apparatus as in claim 12, further comprising a belt, wherein said handle slides onto said belt.

17. A method of spinal decompression comprising:
putting on spinal decompression belt;
placing hands on handles of spinal compression belt;
pressing down on handles with handles while keeping shoulders high; and
bringing chest upward.

18. A method as in claim 17, further comprising the step of laying down after releasing the handles.

19. A method as in claim 17, wherein a user repeats the method multiple times throughout the day.

20. A method as in claim 17, further comprising sliding a handles on a belt to create the spinal decompression belt.
